(19) United States
(12) Patent Application Publication
(10) Pub. No.: US 2009/0182258 A1
(43) Pub. Date: Jul. 16, 2009

(54) OXIMETRY AND CAPNOGRAPHY SYSTEM FOR USE IN CONNECTION WITH AN EXTRACORPOREAL CIRCULATION PROCEDURE

(76) Inventors: Osvaldo Sobrenome NOGUEIRA SANCHES, Sao Paulo (BR); Nadia Maria Rocha Mendes, Sao Paulo (BR); Gilberto Sobrenome De Oliveira Scuciato, Sao Paulo (BR)

Correspondence Address: SUGHRUE MION, PLLC, 2100 PENNSYLVANIA AVENUE, N.W., SUITE 800, WASHINGTON, DC, 20037, US

(21) Appl. No.: 12/410,970

(22) Filed: Mar. 25, 2009

Related U.S. Application Data
(63) Continuation-in-part of application No. 11/923,101, filed on Oct. 24, 2007.

(30) Foreign Application Priority Data
Nov. 27, 2006 (BR) 0605449-8

(51) Int. Cl. A61M 1/36

(52) U.S. Cl. 604/4.01

(57) ABSTRACT

An oximetry and capnography monitoring system associated to a gas analyzer applied to an ECC procedure, in which a mechanical device based on a tubular circuit is contemplated, such that when the device is connected to the ECC equipment with the main purposes of monitoring the operation of the gas blender, analyzing the oxygenation chamber performance, and checking carbon dioxide outflow from the oxygenation chamber in such a way to prevent collapse within the oxygenation chamber and therefore increasing efficiency throughout the ECC procedure.

This is a request for a Continuation-in-Part Application of pending prior application Ser. No. 11/923,101, filed Oct. 24, 2007 which claims priority to Brazilian Patent Application No. 0605449-8, filed Nov. 27, 2006. The entire disclosures of the prior applications are considered part of the disclosure of the accompanying continuation-in-part application and are hereby incorporated by reference.

### FIELD OF THE INVENTION

The present invention relates to extracorporeal circulation procedures.

### BACKGROUND OF THE INVENTION

The present invention is directed at an oximetry and capnography system applied to an extracorporeal circulation procedure (hereinafter referred to as “ECC”) and, more specifically, to a gas monitoring system for use during an extracorporeal circulation procedure. The present invention includes acknowledging the functioning of the gas mixer (blender), performance analysis of the oxygenation chamber, gauging carbon dioxide output content of the oxygenation chamber, preventing collapse of the oxygenation chamber and increasing efficiency of the entire ECC procedure.

Membrane collapse occurs when the pressure of the injected gas in the oxygenator is equal to atmospheric pressure, which impedes gas circulation inside the membrane, the gas remaining in inertia. In other words, when the gas is hindered from leaving the oxygenator due to atmospheric air pressure, membrane collapse results.

As is known by skilled technicians in the art, extracorporeal circulation (ECC) is a surgical procedure employed in certain operations, particularly those involving the heart, where the patient's blood is taken out of the body for purification and oxygenation, after which it is returned to the body. Purification and oxygenation are performed by equipment that provides an artificial substitute for the patient's heart and lung. A pump performs the work of the heart by pumping blood through a circuit of medical tubing attached to the patient's vascular system, while an oxygenator, including a gas mixer (blender), performs the work of the lung by oxygenating the blood before returning it to the patient.

This equipment is monitored by perfusionists who are professionals with knowledge of physiopathology, perfusion and the necessary surgical techniques to perform the procedure. Among other functions, the perfusionist is responsible for adequately monitoring blood gasometry, which involves constant analysis of data collected by the various pieces of equipment, and correcting, if necessary, oxygen and carbonic gas concentrations in the patient's bloodstream, based on the collected data.

Generally, perfusionists act based on their own professional training and experience by empirically verifying the quality of the blood that leaves the patient and the quality of the blood that is returned to the patient, a process known by professionals in the field as “arteriovenous difference”.

In the event that any of the analysis equipment functions erroneously or fails entirely, the patient's life is immediately placed at risk. Accordingly, it is of the utmost importance to verify the gasometry of the blood in an accurate and reliable manner. Therefore, upon recognition of any deviation in the measurements provided by the analytical equipment, one can proceed to check the equipment, preventing any problem for the patient.

U.S. patent application Ser. No. 11/923,101 provided a system that monitors, through oximetry and capnography in association with a gas analyzer, the functioning of the equipment during EEC, permitting the identification of any discrepancies or impending problems during the procedure, which can therefore be solved immediately, preventing inconveniences during surgery. The Application presents an oximetry and capnography system applied to an extracorporeal circulation (ECC) procedure. More specifically, teaches an oximetry and capnography monitoring system employing a gas analyzer applied in extracorporeal circulation. A mechanical device based on a circuit of tubes and connectors that, when properly installed in connection with the ECC equipment, monitors the functioning of a gas blender (gas mixer), analyzes the performance of the oxygenation chamber of an oxygenator, gauges the carbon dioxide output content (or outflow) of the oxygenation chamber (preventing collapse of the oxygenation chamber), which increases the efficiency of the entire ECC procedure.

These particular advantages, along with other objectives and advantages presented by U.S. patent application Ser. No. 11/923,101 are attained by the oximetry and capnography system for use in an ECC procedure, where a gas sample is taken from the oxygenator and transmitted via a tube to the gas analyzer, which constantly verifies the concentration of the sample, while simultaneously checking the existing concentration of the gas blender.

Oxygenation performance is evaluated through measurement of the gas input and output in the oxygenator. For this reason, only one gas output of the oxygenator is kept open, while the others are closed.

The system taught in U.S. patent application Ser. No. 11/923,101 permits control (through capnography) of carbon dioxide output from the oxygenator, such that its removal is determined by gas flow in relation to arterial flow.

It is well known that patient recovery after an ECC procedure associated with treatment for hypothermia is slow, particularly due to the vasoconstriction caused by temperature reduction of the patient's vascular tissues.

Hypothermia is necessary in order to reduce the patient's metabolism (by decreasing oxygen consumption) which protects the organism during periods of reduced blood flow, such as during extracorporeal circulation procedures. Induced hypothermia reduces metabolic needs and permits periods of low flow or circulatory arrest to be prolonged within safety limits. Dispite of these advantages, hypothermia is a significant physiological deviation of the patient's body temperature, which naturally maintains a body temperature of around 37° C.

Upon lowering body temperature, the solubility of oxygen and carbon dioxide in the patient's blood stream increases, while reducing patient metabolism decreases the production of carbon dioxide, which contracts the blood vessels. Cerebral auto-regulation, which is the mechanism that maintains constant cerebral blood flow, disappears in patients with temperature below 28° C. As the patient's body temperature lowers, resistance increases (due to constricted vascular tissues), which decreases cerebral blood flow.

In special situations, such as the treatment of newborns where the oxygenation chamber is incompatible with the child's body surface (i.e.: the large equipment is impractical for a small child) or where hypothermic patients undergo an ECC procedure, an exaggerated and persistent drop in the partial arterial carbon dioxide (PaCO₂) pressure is observed due to decreased cellular metabolism in the patient.

Therefore, in order to prevent this from occurring the present invention provides an improvement in the oximetry and capnography systems applied to extracorporeal circulation procedures, whereby carbon dioxide is injected in the extracorporeal circulation to promote vasodilatation, preserving cerebral blood flow during cooling and in the reheating of the patient, thereby permitting faster normalization of cerebral blood flow after the ECC procedure.

### BRIEF DESCRIPTION OF THE DRAWINGS

FIG. 1 is a schematic view of the oximetry and capnography system.

### SUMMARY OF THE INVENTION

The present application seeks to provide an oximetry and capnography system for use in connection with an extracorporeal circulation procedure, the system comprising; (a) an oxygenator, having an oxygenation chamber, a first blood input, a first blood output, a second blood input, a second blood output, and a carbon dioxide supply line; (b) means for connecting the oxygenator to a vascular system of a surgical patient, including an inflow connecting means and an outflow connecting means; (c) an extracorporeal circulation machine, connected to the oxygenator; (d) a gas blender, connected to the oxygenator and to the carbon dioxide supply line; (e) a carbon dioxide supply vessel including a flow meter, connected to the gas blender and connected to the carbon dioxide supply line through a Y-shaped connector; (f) a blood sampling device, including a first blood sampling circuit and a second blood sampling circuit, the first and second sampling circuits connected to the oxygenator, and (g) a gas analyzer, connected to the gas blender and to the oxygenator through the first blood sampling device and the second blood sampling device.

The venous blood is conveyed from the vascular system of the surgical patient to the oxygenator first blood input through the inflow connecting means; the venous blood exits the oxygenator through the first blood output and is conveyed to the extracorporeal circulation machine; the extracorporeal circulation machine receives the venous blood and conveys the venous blood back to the oxygenator through the oxygenator second blood input, creating an extracorporeal circulation; a first gas sample is removed from the second blood input of the oxygenator by the first circuit of the blood sampling device and is conveyed to the gas analyzer; the oxygenator oxygenates the blood in the oxygenation chamber; a second gas sample is removed from the second blood output of the oxygenator by the second circuit of the blood sampling device and is conveyed to the gas analyzer such that efficiency and performance of the oximetry and capnography system is measured through a comparison of the input gas sample and the output gas sample. The carbon dioxide supply vessel simultaneously provides a predetermined amount of the carbon dioxide to the gas blender, and the gas blender blends the predetermined amount of the carbon dioxide into the system through the oxygenator carbon dioxide supply line; the predetermined amount of carbon dioxide controlled through oximetry or capnography, and the arterial blood is conveyed from the second blood output of the oxygenator to the vascular system of the surgical patient through the outflow connecting means.

### DETAILED DESCRIPTION OF THE INVENTION

The invention will be described for the purposes of illustration only in connection with certain embodiments; however, it is to be understood that other objects and advantages of the present invention will be made apparent by the following description of the drawings according to the present invention. While a preferred embodiment is disclosed, this is not intended to be limiting. Rather, the general principles set forth herein are considered to be merely illustrative of the scope of the present invention and it is to be further understood that numerous changes may be made without straying from the scope of the present invention.

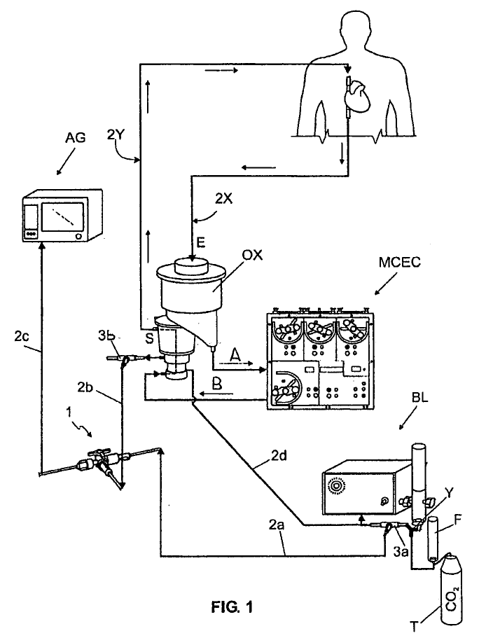

As illustrated in FIG. 1, the oximetry and capnography system applied to extracorporeal circulation procedure incorporates a first blood sampling mechanical device (1) formed by two circuits with flexible tubes (2a, 2b, 2c and 2d) and connectors (3a and 3b) that perform constant monitoring of the extracorporeal circulation.

The venous blood leaves the heart of a surgical patient and is conveyed through the tube (2X) to the oxygenator (OX). The gas enters the oxygenator at the first blood input (E) and from the oxygenator the blood is conveyed to the ECC machine (MCEC) through first blood output (A). From the MCEC machine, a propulsion pump (not shown) returns the blood to the oxygenator (OX) through second blood input (B), where the blood is oxygenated and transformed into arterial blood for return to the patient through the second blood output (S) and the tube (2Y). This way it is created an extracorporeal circulation.

Blood sampling mechanical device (1), includes a first circuit equipped with the connector (3a) connected to the tube (2a), which carries a gas sample that is entering into the oxygenator (OX) to the sampling device (1) (connection not shown), which, connected to the tube (2C), conveys the gas sample to the gas analyzer (AG). The gas analyzer constantly checks the real concentration, and simultaneously checks with the existing concentration in the gas blender (BL).

In a second circuit of the blood sampling mechanical device (1) connector (3b) located at the oxygenator (OX) second output (S), takes a sample of the gas exiting the oxygenator (OX), conveys the sample to the mechanical device (1) and through the tube (2b), conveys the sample to the gas analyzer (AG), checking the carbon dioxide outflow from the oxygenator (OX) due to venous blood change to arterial blood. The gas analyzer (AG) analyzes oxygenation performance.

The system analyzes the oximetry of the oxygenator. Control through capnography is performed at the carbon dioxide output through the oxygenator (OX), where carbon dioxide removal is related with gas flow in relation to arterial flow. By means of oximetry and capnography, the system analyzes the gas blender (BL) and gas exchange system efficiency within the oxygenation chamber of the single-exit oxygenator through the comparison of the gas entering at the first blood input (E) the oxygenator (OX) with the gas exiting the second blood output (S) of the oxygenator (OX). The term single-exit in the present invention means having a unique exit from the oxygenator to the surgical patient.

As it can also be seen in FIG. 1, a flow meter (F) is attached to the gas blender (BL), for introduction of carbon dioxide, which then continues through a tube gas line (2d), which is a carbon dioxide supply line to the oxygenator (OX). The carbon dioxide, together with oxygen, is released in the arterial blood to the surgical patient.

A carbon dioxide torpedo (T), which is a carbon dioxide supply vessel, is used to apply the carbon dioxide to the flow meter (F), which is connected through a Y-shaped connector (Y).

Therefore, it is possible to maintain an adequate partial arterial carbon dioxide (PaCO₂) pressure, for example, of 40 mmHg, regardless of the patient's temperature, with the introduction of carbon dioxide in the oxygenation system, which also provides carbon dioxide to the patient's blood.

The control of carbon dioxide in the oxygenation chamber is done through a combination of the gas blender (BL) and with the 100% carbon dioxide torpedo (T) in communication with the oxygenation line (2d) of the membrane oxygenator (OX).

When the arterial blood presents a PaCO₂ below to what is desired, the oxygenation chamber, besides oxygenating, transfers the carbon dioxide to the blood, increasing arterial PaCO₂. This prevents the contraction of blood vessels, permitting the patient's faster recovery at the time the body temperature is returning to normal.

For this technique may be employed with efficiency and with reliability in the ECC, the control of gases has to be gauged both at the input (E) as well as at the output (S) of the membrane oxygenator (OX), such that the oxygenator (OX) must present a single gas output in the oxygenation chamber.

The quantity of carbon dioxide injected in the system is normally controlled by the PaCO₂ value during ECC. This control is done through the ratio of arterial flow with the gas flow of the gas blender (BL), and the lesser is the gas flow in relation to arterial flow, the greater is PaCO₂ in the blood, and vice versa. Being insufficient the control of gas flow through oximetry, its analysis through capnography is done at the output of the oxygenation chamber.

The system of the present invention maintains a concentration of carbon dioxide in the oxygenator chamber (OX), preserving an adequate PaCO₂ in the arterial blood (PaCO₂ of 40 mmHg), even with low cellular metabolism due to the patient's hypothermia.

This usage shows that PaCO₂ influences significantly cerebral flow and the peripheral system, and therefore the carbon dioxide employed in adequate quantity in the arterial blood results in a significant increase in cerebral blood flow.

1. An oximetry and capnography system for use in connection with an extracorporeal circulation procedure, the system comprising;
(a) an oxygenator, having an oxygenation chamber, a first blood input, a first blood output, a second blood input, a second blood output, and a carbon dioxide supply line;
(b) means for connecting the oxygenator to a vascular system of a surgical patient, including an inflow connecting means and an outflow connecting means;
(c) an extracorporeal circulation machine, connected to the oxygenator;
(d) a gas blender, connected to the oxygenator and to the carbon dioxide supply line;
(e) a carbon dioxide supply vessel including a flow meter, connected to the gas blender and connected to the carbon dioxide supply line through a Y-shaped connector;
(f) a blood sampling device, including a first blood sampling circuit and a second blood sampling circuit, the first and second sampling circuits connected to the oxygenator, and
(g) a gas analyzer, connected to the gas blender and to the oxygenator through the first blood sampling device and the second blood sampling device,
wherein venous blood is conveyed from the vascular system of the surgical patient to the oxygenator first blood input through the inflow connecting means; the venous blood exits the oxygenator through the first blood output and is conveyed to the extracorporeal circulation machine; the extracorporeal circulation machine receives the venous blood and conveys the venous blood back to the oxygenator through the oxygenator second blood input, creating an extracorporeal circulation; a first gas sample is removed from the second blood input of the oxygenator by the first circuit of the blood sampling device and is conveyed to the gas analyzer; the oxygenator oxygenates the blood in the oxygenation chamber; a second gas sample is removed from the second blood output of the oxygenator by the second circuit of the blood sampling device and is conveyed to the gas analyzer such that efficiency and performance of the oximetry and capnography system is measured through a comparison of the input gas sample and the output gas sample, and
wherein the carbon dioxide supply vessel simultaneously provides a predetermined amount of the carbon dioxide to the gas blender, and the gas blender blends the predetermined amount of the carbon dioxide into the system through the oxygenator carbon dioxide supply line; the predetermined amount of carbon dioxide controlled through oximetry or capnography, and
wherein the arterial blood is then conveyed from the second blood output of the oxygenator to the vascular system of the surgical patient through the outflow connecting means.
